(19) United States
(12) Patent Application Publication
(10) Pub. No.: US 2004/0087792 A1
(43) Pub. Date: May 6, 2004

(54) Tryptase-inhibitors

(76) Inventors: Thomas Martin, Konstanz (DE); Wolf-Rudiger Ulrich, Konstanz (DE)

Correspondence Address: Nath & Associates, Sixth Floor, 1030 15th Street NW, Washington, DC, 20005, US

(21) Appl. No.: 10/469,755

(22) PCT Filed: Mar. 12, 2002
(86) PCT No.: PCT/EP02/02675

(30) Foreign Application Priority Data
Mar. 15, 2001 (EP) 01106541.4

(51) Int. Cl. C07C 69/76; C07D 213/55; C07D 241/02

(52) U.S. Cl. 544/336; 546/341; 560/105

(57) ABSTRACT

Compounds of the formula I, wherein M, A1, A2, K1 and K2 have the meanings as indicated in the description are novel effective tryptase inhibitors.

### FIELD OF APPLICATION OF THE INVENTION

The invention relates to novel tryptase inhibitors which are used in the pharmaceutical industry for preparing medicaments.

### KNOWN TECHNICAL BACKGROUND

The international applications WO95/32945, WO96/09297, WO98/04537, WO99/12918, WO99/24395, WO99/24407, WO99/40073, WO00/14097 and WO01/10848 describe low-molecular-weight bivalent compounds for use as tryptase inhibitors.

### DESCRIPTION OF THE INVENTION

It has now been found that the compounds of the formula I, which are described in more detail below, have surprising and particularly advantageous properties.

The invention provides compounds of the formula I

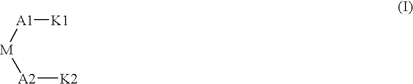

in which

M is a central building block selected from the formulae below

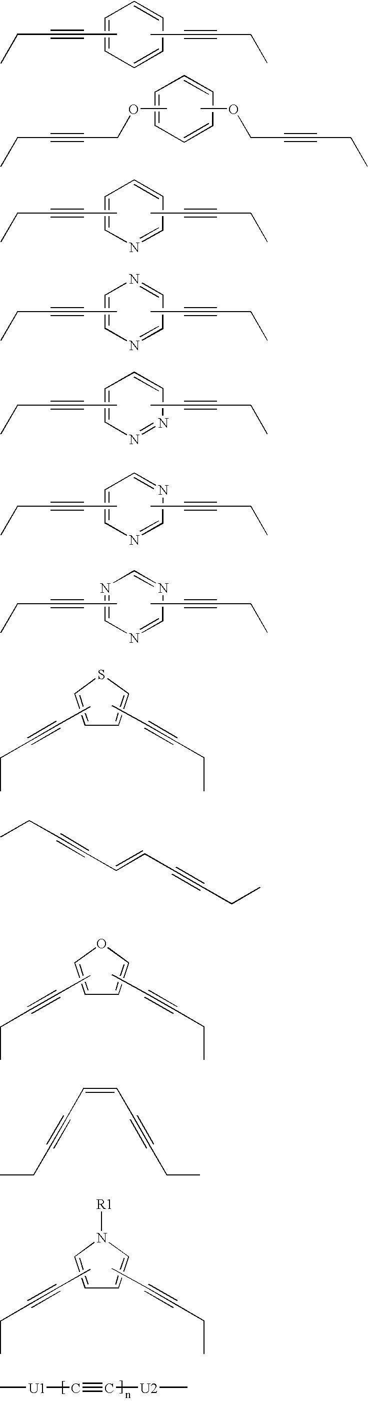

wherein

R1 is hydrogen, 1-4C-alkyl or 1-4C-alkylcarbonyl,

n is 1 or 2,

U1 and U2 are identical or different and are methylene [—CH₂—], ethylene [—CH₂—CH₂—], trimethylene [—CH₂—CH₂—CH₂—], tetramethylene [—CH₂—CH₂—CH₂—CH₂—] or isopropylidene [—C(CH₃ )₂—],

A1 is -A3-B1-A5-,

A2 is -A4-B2-A6-,

wherein either

A3 is —C(O)—, —O—C(O)—, —C(O)—NH—, —NH—C(O)—, —O—C(O)—NH—, —NH—C(O)—O—, —NH—C(O)—NH—, —O—C(O)—O—, —O—(CH₂)ᵣ—C(O)—, —O—(CH₂)ᵣ—C(O)—NH—, —O—(CH₂)ₘ—O—C(O)— or —O—(CH₂)ₘ—NH—C(O)—,

A4 is —C(O)—, —O—C(O)—, —C(O)—NH—, —NH—C(O)—, —O—C(O)—NH—, —NH—C(O)—O—, —NH—C(O)—NH—, —O—C(O)—O—, —O—(CH₂)ᵣ—C(O)—, —O—(CH₂)ᵣ—C(O)—NH—, —O—(CH₂)ₘ—O—C(O)— or —O—(CH₂)ₘ—NH—C(O)—,

A5 is —C(O)—N(R2)-, —N(R2)-C(O)—, —O—C(O)—N(R2)- or —N(R2)-C(O)—O—, and

A6 is —C(O)—N(R3)-, —N(R3)-C(O)—, —O—C(O)—N(R3)- or —N(R3)-C(O)—O—,

or

A3 is —C(O)—N(R4)-, —N(R4)-C(O)—, —O—C(O)—N(R4)-, —N(R4)-C(O)—O—, —O—(CH₂)ᵣ—C(O)—N(R4)- or —O—(CH₂)ₘ—N(R4)-C(O)—,

A4 is —C(O)—N(R5)-, —N(R5)-C(O)—, —O—C(O)—N(R5)-, —N(R5)-C(O)—O—, —O—(CH₂)ᵣ—C(O)—N(R5)- or —O—(CH₂)ₘ—N(R5)-C(O)—,

A5 is —C(O)—, —C(O)—NH—, —NH—C(O)—, —O—C(O)—NH—, —NH—C(O)—O—, —NH—C(O)—NH— or —O—C(O)—O—, and

A6 is —C(O)—, —C(O)—NH—, —NH—C(O)—, —O—C(O)—NH—, —NH—C(O)—O—, —NH—C(O)—NH— or —O—C(O)—O—,

or

A3 is —C(O)—N(R4)-, —N(R4)-C(O)—, —O—C(O)—N(R4)-, —N(R4)-C(O)—O—, —O—(CH₂)ᵣ—C(O)—N(R4)- or —O—(CH₂)ₘ—N(R4)-C(O)—,

A4 is —C(O)—, —O—C(O)—, —C(O)—NH—, —NH—C(O)—, —O—C(O)—NH—, —NH—C(O)—O—, —NH—C(O)—NH—, —O—C(O)—O—, —O—(CH₂)ᵣ—C(O)—, —O—(CH₂)ᵣ—C(O)—NH—, —O—(CH₂)ₘ—O—C(O)— or —O—(CH₂)ₘ—NH—C(O)—,

A5 is —C(O)—N(R2)-, —N(R2)-C(O)—, —O—C(O)—N(R2)- or —N(R2)-C(O)—O—, and

A6 is —C(O)—, —C(O)—NH—, —NH—C(O)—, —O—C(O)—NH—, —NH—C(O)—O—, —NH—C(O)—NH— or —O—C(O)—O—,

or

A3 is —C(O)—, —O—C(O)—, —C(O)—NH—, —NH—C(O)—, —O—C(O)—NH—, —NH—C(O)—O—, —NH—C(O)—NH—, —O—C(O)—O—, —O—(CH₂)ᵣ—C(O)—, —O—(CH₂)ᵣ—C(O)—NH—, —O—(CH₂)ₘ—O—C(O)— or —O—(CH₂)ₘ—NH—C(O)—,

A4 is —C(O)—N(R5)-, —N(R5)-C(O)—, —O—C(O)—N(R5)-, —N(R5)-C(O)—O—, —O—(CH₂)ᵣ—C(O)—N(R5)- or —O—(CH₂)ₘ—N(R5)-C(O)—,

A5 is —C(O)—, —C(O)—NH—, —NH—C(O)—, —O—C(O)—NH—, —NH—C(O)—O—, —NH—C(O)—NH— or —O—C(O)—O—, and

A6 is —C(O)—N(R3)-, —N(R3)-C(O)—, —O—C(O)—N(R3)- or —N(R3)-C(O)—O—,

or

A3 is —C(O)—N(R4)-, —N(R4)-C(O)—, —O—C(O)—N(R4)-, —N(R4)-C(O)—O—, —O—(CH₂)ᵣ—C(O)—N(R4)- or —O—(CH₂)ₘ—N(R4)-C(O)—,

A4 is —C(O)—, —O—C(O)—, —C(O)—NH—, —NH—C(O)—, —O—C(O)—NH—, —NH—C(O)—O—, —NH—C(O)—NH—, —O—C(O)—O—, —O—(CH₂)ᵣ—C(O)—, —O—(CH₂)ᵣ—C(O)—NH—, —O—(CH₂)ₘ—O—C(O)— or —O—(CH₂)ₘ—NH—C(O)—,

A5 is —C(O)—, —C(O)—NH—, —NH—C(O)—, —O—C(O)—NH—, —NH—C(O)—O—, —NH—C(O)—NH— or —O—C(O)—O—, and

A6 is —C(O)—N(R3)-, —N(R3)-C(O)—, —O—C(O)—N(R3)- or —N(R3)-C(O)—O—,

or

A3 is —C(O)—, —O—C(O)—, —C(O)—NH—, —NH—C(O)—, —O—C(O)—NH—, —NH—C(O)—O—, —NH—C(O)—NH—, —O—C(O)—O—, —O—(CH₂)ᵣ—C(O)—, —O—(CH₂)ᵣ—C(O)—NH—, —O—(CH₂)ₘ—O—C(O)— or —O—(CH₂)ₘ—NH—C(O)—,

A4 is —C(O)—N(R5)-, —N(R5)-C(O)—, —O—C(O)—N(R5)-, —N(R5)-C(O)—O—, —O—(CH₂)ᵣ—C(O)—N(R5)- or —O—(CH₂)ₘ—N(R5)-C(O)—,

A5 is —C(O)—N(R2)-, —N(R2)-C(O)—, —O—C(O)—N(R2)- or —N(R2)-C(O)—O—, and

A6 is —C(O)—, —C(O)—NH—, —NH—C(O)—, —O—C(O)—NH—, —NH—C(O)—O—, —NH—C(O)—NH— or —O—C(O)—O—,

and

r is1, 2, 3or4,

m is1, 2, 3or4,

R2, R3, R4 and R5 are identical or different and are —CH₂—C(O)OR6 or —CH₂—C(O)N(R7)R8,

R6 is hydrogen, 1-4C-alkyl, 3-7C-cycloalkyl, 3-7C-cycloalkylmethyl or benzyl,

R7 and R8 are independent from each other hydrogen, 1-4C-alkyl, 3-7C-cycloalkyl, 3-7C-cycloalkylmethyl, or wherein R7 and R8 together and with inclusion of the nitrogen atom to which they are bonded form a 1-pyrrolidinyl-, 1-piperidinyl-, 1-hexahydroazepinyl-, 1-piperazinyl- or 4-morpholinyl-radical,

B1 and B2 are identical or different and are 1-4C-alkylene, 1,4-cyclohexylene, 1,3-cyclohexylene, 1,4-phenylene, 1,3-phenylene, 1,4-piperazinylene or 1,4-piperidinylene,

K1 is -B3-X1, -B3-Y1 or -B3-Z1-B5-X1,

K2 is -B4-X2, -B4-Y2 or -B4-Z2-B6-X2,

B3 and B4 are identical or different and are a bond or 1-4C-alkylene,

B5 and B6 are identical or different and are a bond or 1-2C-alkylene,

X1 and X2 are identical or different and are amino, aminocarbonyl or amidino,

Y1 and Y2 are imidazol-1-yl,

Z1 and Z2 are identical or different and are 5,2-pyridinylene, 6-methyl-5,2-pyridinylene, 4,1-piperidinylene, 3,6-indazolylene, 3,6-indolylene, 1,3-phenylene, 1,4-phenylene, 1,3-cyclohexylene or 1,4-cyclohexylene,

the salts of these compounds, and the N-oxides of the nitrogen-containing heteroaryls, heteroarylenes and heterocycloalkylenes, and their salts, where all those compounds are excluded in which, owing to the meaning of the variables A3, A4, A5, A6, B1, B2, B3, B4, Y1, Y2, Z1, Z2, X1 or X2, there would be a direct linkage of two heteroatoms or two carbonyl groups.

1-4C-Alkyl represents straight-chain or branched alkyl radicals having 1 to 4 carbon atoms. Examples which may be mentioned are the butyl, isobutyl, sec-butyl, tert-butyl, propyl, isopropyl, ethyl and the methyl radicals.

3-7C-Cycloalkyl represents cyclopropyl, cyclobutyl, cyclopentyl, cyclohexyl and cycloheptyl, of which cyclopropyl, cyclobutyl and cyclopentyl are preferred.

3-7C-Cycloalkylmethyl represents a methyl radical which is substituted by one of the abovementioned 3-7C-cycloalkyl radicals. The 3-5C-cycloalkylmethyl radicals cyclopropylmethyl, cyclobutylmethyl and cyclopentylmethyl may be mentioned preferably.

1-4C-alkylene represents straight-chain or branched 1-4C-alkylene radicals, for example the methylene [—CH₂—], ethylene [—CH₂—CH₂—], trimethylene [—CH₂—CH₂—CH₂—], tetramethylene [—CH₂—CH₂—CH₂—CH₂—], 1,2-dimethylethylene [—CH(CH₃)—CH(CH₃)—], 1,1-dimethylethylene [—C(CH₃)₂—CH₂—], 2,2-dimethylethylene [—CH₂—C(CH₃)₂—], isopropylidene [—C(CH₃)₂—] or the 1-methylethylene [—CH(CH₃)—CH₂—] radicals.

1-4C-Alkylcarbonyl represents a radical which, in addition to the carbonyl group, contains one of the abovementioned 1-4C-alkyl radicals. An example which may be mentioned is the acetyl radical.

By definition, the groups Z1 and Z2 are located between groups B3 and B5 (-B3-Z1-B5-) and B4 and B6 (-B4-Z2-B6-), respectively. Accordingly, in the divalent groupings mentioned by way of example (for example 3,6-indolylene), the first number indicates the point of attachment to the group B3 and B4, respectively, and the second number indicates the point of attachment to the group B5 and B6, respectively.

The definition of M contains chemical formulae, such as, for example,

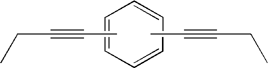

In the context of this invention this means that the two —CH≡CH—CH₂— groups can be bonded to the phenyl ring in 1,2-, 1,3- and 1,4-position, of which the 1,3- and 1,4-position are preferred. The same principle has to be applied for the other chemical formulae which are part of the definition of M.

Suitable salts for compounds of the formula I—depending on substitution—are all acid addition salts or all salts with bases. Particular mention may be made of the pharmacologically tolerable salts of the inorganic and organic acids and bases customarily used in pharmacy. Those which are suitable are, on the one hand, water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methane sulfonic acid or 3-hydroxy-2-naphthoic acid, where the acids are employed in salt preparation—depending on whether it is a mono- or polybasic acid and depending on which salt is desired—in an equimolar quantitative ratio or one differing therefrom.

On the other hand, salts with bases are also suitable. Examples of salts with bases which may be mentioned are alkali metal (lithium, sodium, potassium) or calcium, aluminum, magnesium, titanium, ammonium, meglumine or guanidinium salts, where here too the bases are employed in salt preparation in an equimolar quantitative ratio or one differing therefrom.

Pharmacologically unacceptable salts which can be obtained initially as process products, for example in the preparation of the compounds according to the invention on an industrial scale, are converted into pharmacologically acceptable salts by processes known to the person skilled in the art.

It is known to the person skilled in the art that the compounds according to the invention, and also their salts, may contain varying amounts of solvents, for example when they are isolated in crystalline form. The invention therefore also embraces all solvates and in particular all hydrates of the compounds of the formula I, and also all solvates and in particular all hydrates of the salts of the compounds of the formula I.

Compounds of the formula I which are to be emphasized are those in which M is a central building block selected from the formulae below

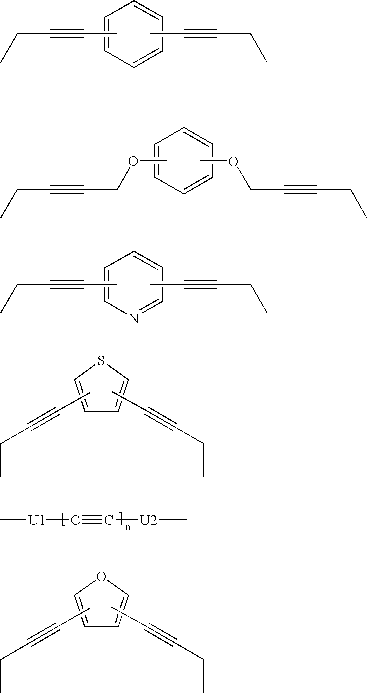

wherein

n is 1 or2,

U1 and U2 are identical or different and are methylene [—CH₂—], ethylene [—CH₂—CH₂—], trimethylene [—CH₂—CH₂—CH₂—], tetramethylene [—CH₂—CH₂—CH₂—CH₂—] or isopropylidene [—C(CH₃)₂—],

A1 is -A3-B1-A5-,

A2 is -A4-B2-A6-,

wherein either

A3 is —O—C(O)—, —NH—C(O)—, —O—C(O)—NH—, —NH—C(O)—O—, —NH—C(O)—NH—, —O—C(O)—O—, —O—(CH₂)ᵣ—C(O)—, —O—(CH₂)ᵣ—C(O)—NH—, —O—(CH₂)ₘ—O—C(O)— or —O—(CH₂)ₘ—NH—C(O)—,

A4 is —O—C(O)—, —NH—C(O)—, —O—C(O)—NH—, —NH—C(O)—O—, —NH—C(O)—NH—, —O—C(O)—O—, —O—(CH₂)ᵣ—C(O)—, —O—(CH₂)ᵣ—C(O)— or —O—(CH₂)ₘ—NH—C(O)—,

A5 is —C(O)—N(R2)- or —N(R2)-C(O)—, and

A6 is —C(O)—N(R3)- or —N(R3)-C(O)—,

or

A3 is —N(R4)-C(O)—, —O—C(O)—N(R4)-, —N(R4)-C(O)—O—, —O—(CH₂)ᵣ—C(O)—N(R4)- or —O—(CH₂)ₘ—N(R4)-C(O)—,

A4 is —N(R5)-C(O)—, —O—C(O)—N(R5)-, —N(R5)-C(O)—O—, —O—(CH₂)ᵣ—C(O)—N(R5)- or —O—(CH₂)ₘ—N(R5)-C(O)—,

A5 is —C(O)—, —C(O)—NH— or —NH—C(O)—, and

A6 is —C(O)—, —C(O)—NH— or —NH—C(O)—,

or

A3 is —C(O)—N(R4)-, —N(R4)-C(O)—, —O—C(O)—N(R4)-, —N(R4)-C(O)—O—, —O—(CH₂)ᵣ—C(O)—N(R4)- or —O—(CH₂)ₘ—N(R4)-C(O)—,

A4 is —O—C(O)—, —NH—C(O)—, —O—C(O)—NH—, —NH—C(O)—O—, —NH—C(O)—NH—, —O—C(O)—O—, —O—(CH₂)ᵣ—C(O)—, —O—(CH₂)ᵣ—C(O)—NH—, —O—(CH₂)ₘ—O—C(O)— or —O—(CH₂)ₘ—NH—C(O)—,

A5 is —C(O)—N(R2)- or —N(R2)-C(O)—, and

A6 is —C(O)—, —C(O)—NH— or —NH—C(O)—,

or

A3 is —O—C(O)—, —NH—C(O)—, —O—C(O)—NH—, —NH—C(O)—O—, —NH—C(O)—NH—, —O—C(O)—O—, —O—(CH₂)ᵣ—C(O)—, —O—(CH₂)ᵣ—C(O)—NH—, —O—(CH₂)ₘ—O—C(O)— or —O—(CH₂)ₘ—NH—C(O)—,

A4 is —N(R5)-C(O)—, —O—C(O)—N(R5)-, —N(R5)-C(O)—O—, —O—(CH₂)ᵣ—C(O)—N(R5)- or —O—(CH₂)ₘ—N(R5)-C(O)—,

A5 is —C(O)—, —C(O)—NH— or —NH—C(O)—, and

A6 is —C(O)—N(R3)- or —N(R3)-C(O)—,

or

A3 is —N(R4)-C(O)—, —O—C(O)—N(R4)-, —N(R4)-C(O)—O—, —O—(CH₂)ᵣ—C(O)—N(R4)- or —O—(CH₂)ₘ—N(R4)-C(O)—,

A4 is —O—C(O)—, —NH—C(O)—, —O—C(O)—NH—, —NH—C(O)—O—, —NH—C(O)—NH—, —O—C(O)—O—, —O—(CH₂)ᵣ—C(O)—, —O—(CH₂)ᵣ—C(O)—NH—, —O—(CH₂)ₘ—O—C(O)— or —O—(CH₂)ₘ—NH—C(O)—,

A5 is —C(O)—, —C(O)—NH— or —NH—C(O)—, and

A6 is —C(O)—N(R3)- or —N(R3)-C(O)—,

or

A3 is —O—C(O)—, —NH—C(O)—, —O—C(O)—NH—, —NH—C(O)—O—, —NH—C(O)—NH—, —O—C(O)—O—, —O—(CH₂)ᵣ—C(O)—,—O—(CH₂)ᵣ—C(O)—NH—, —O—(CH₂)ₘ—O—C(O)— or —O—(CH₂)ₘ—NH—C(O)—,

A4 is —N(R5)-C(O)—, —O—C(O)—N(R5)-, —N(R5)-C(O)—O—, —O—(CH₂)ᵣ—C(O)—N(R5)- or —O—(CH₂)ᵣ—N(R5)-C(O)—,

A5 is —C(O)—N(R2)- or —N(R2)-C(O)—, and

A6 is —C(O)—, —C(O)—NH— or —NH—C(O)—,

and

r is 1 or 2,

m is 2,

R2, R3, R4 and R5 are identical or different and are —CH₂—C(O)OR6 or —CH₂—C(O)N(R7)R8,

R6 is hydrogen, 1-4C-alkyl, 3-7C-cycloalkyl, 3-7C-cycloalkylmethyl or benzyl,

R7 and R8 are independent from each other hydrogen, 1-4C-alkyl, 3-7C-cycloalkyl, 3-7C-cycloalkylmethyl, or wherein R7 and R8 together and with inclusion of the nitrogen atom to which they are bonded form a 1-pyrrolidinyl-, 1-piperidinyl-, 1-hexahydroazepinyl-, 1-piperazinyl- or 4-morpholinyl-radical,

B1 and B2 are identical or different and are 1-4C-alkylene, 1,4-cyclohexylene, 1,3-cyclohexylene, 1,4-phenylene, 1,3-phenylene, 1,4-piperazinylene or 1,4-piperidinylene,

K1 is -B3-Z1-B5-X1,

K2 is -B4-Z2-B6-X2,

B3 and B4 are identical or different and are a bond or 1-2C-alkylene,

B5 and B6 are identical or different and are a bond or 1-2C-alkylene,

X1 and X2 are identical or different and are amino or amidino,

Z1 and Z2 are identical or different and are 1,3-phenylene, 1,4-phenylene, 1,3-cyclohexylene or 1,4-cyclohexylene,

and the salts of these compounds, and the N-oxides of the nitrogen-containing heterocycloalkylenes, and their salts, where all those compounds are excluded in which, owing to the meaning of the variables A3, A4, A5, A6, B1 or B2, there would be a direct linkage of two heteroatoms.

Compounds of the formula I which are to be particularly emphasized are those in which M is a central building block selected from the formulae below

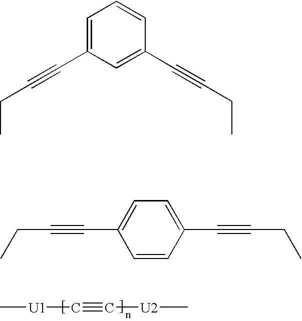

wherein

n is 1 or 2,

U1 and U2 are identical and are methylene [—CH₂—],

A1 is -A3-B1-A5-,

A2 is -A4-B2-A6-,

wherein either

A3 is —O—C(O)—NH—,

A4 is —O—C(O)—NH—,

A5 is —C(O)—N(R2)- or —N(R2)-C(O)—, and

A6 is —C(O)—N(R3)- or —N(R3)-C(O)—,

or

A3 is —O—C(O)—N(R4)-,

A4 is —O—C(O)—N(R5)-,

A5 is —C(O)—NH— or —NH—C(O)—, and

A6 is —C(O)—NH— or —NH—C(O)—,

or

A3 is —O—C(O)—N(R4)-,

A4 is —C(O)—NH—,

A5 is —C(O)—N(R2)- or —N(R2)-C(O)—, and

A6 is —C(O)—NH— or —NH—C(O)—,

or

A3 is —O—C(O)—NH—,

A4 is —O—C(O)—N(R5)-,

A5 is —C(O)—NH— or —NH—C(O)—, and

A6 is —C(O)—N(R3)- or —N(R3)-C(O)—,

or

A3 is —O—C(O)—N(R4)-,

A4 is —O—C(O)—NH—,

A5 is —C(O)—NH— or —NH—C(O)—, and

A6 is —C(O)—N(R3)- or —N(R3)-C(O)—,

or

A3 is —O—C(O)—NH—,

A4 is —O—C(O)—N(R5)-,

A5 is —C(O)—N(R2)- or —N(R2)-C(O)—, and

A6 is —C(O)—NH— or —NH—C(O)—,

and

R2, R3, R4 and R5 are identical and are —CH₂—C(O)OR6,

R6 is hydrogen, 1-4C-alkyl or benzyl,

B1 and B2 are identical and are ethylene,

K1 is -B3-Z1-B5-X1,

K2 is -B4-Z2-B6-X2,

B3 and B4 are identical and are ethylene,

B5 and B6 are identical and are methylene,

X1 and X2 are identical and are amino,

Z1 and Z2 are identical and are 1,3-phenylene or 1,4-phenylene,

and the salts of these compounds.

Preferred compounds of the formula I are those in which

M is a central building block selected from the formulae below

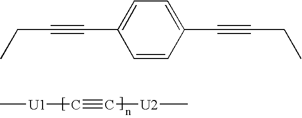

wherein

n is 1,

U1 and U2 are identical and are methylene [—CH₂—],

A1 is -A3-B1-A5-,

A2 is -A4-B2-A6-,

wherein

A3 is —O—C(O)—NH—,

A4 is —O—C(O)—NH—,

A5 is —N(R2)-C(O)—,

A6 is —N(R3)-C(O)—,

R2 and R3 are identical and are —CH₂—C(O)OR6, and

R6 is 1-2C-alkyl,

B1 and B2 are identical and are ethylene,

K1 is -B3-Z1-B5-X1,

K2 is -B4-Z2-B6-X2,

B3 and B4 are identical and are ethylene,

B5 and B6 are identical and are methylene,

X1 and X2 are identical and are amino,

Z1 and Z2 are identical and are 1,4-phenylene,

and the salts of these compounds.

Particularly preferred compounds of the formula I are

1,4-Bis-{N-[3-(4-aminomethyl-phenyl)-propionyl]-N-(ethoxycarbonyl-methyl)-amino-3-(ethyl-aminocarbonyloxy)-prop-1-ynyl}-benzene and

1,4-Bis-{N-[3-(4-(aminomethyl-phenyl)-propionyl]-N-(ethoxycarbonyl-methyl)-amino-3-(ethyl-aminocarbonyloxy)}-2-butyne

and the salts of these compounds.

The compounds of the formula I are constructed from a large number of building blocks (M, A1, A2, A3, A4, A5, A6, B1, B2, B3, B4, B5, B6, X1, X2, Y1, Y2, Z1 and Z2). In principle, they can be synthesized starting with any of these building blocks. If the compounds of the formula I are constructed largely symmetrically, it is favorable to start the synthesis with the central building block M, whereas in the case of predominantly asymmetrical compounds of the formula I a synthesis starting with one of the end groups K1 or K2 may be advantageous.

Suitable starting materials for synthesizing the compounds of the formula I according to the invention are, for example, 1,3-dihydoxybenzene, 1,3-dibromobenzene, 1,4-dibromobenzene, 2,5-dibromofurane, 3,4-dibromothiophen, 2,6-dibromopyridine, 2,5-dibromopyridine, 3,5-dibromopyridine, 2,5-dibromopyridazine, 2,4-dibromopyrimidine, 2,4-dibromo-[1,3,5]-triazine, 2-butyne-1,4-diol, 2,4-hexadiyne-1,6-diol, 2,5-dimethyl-3-hexyne-2,5-diol, 2,7-dimethyl-3,5-octadiyne-2,7-diol or dodec-5,7-diyne-1,12-diol.

Here, the building blocks are linked using always the same pattern, known per se to the person skilled in the art.

It is known to the person skilled in the art that the compounds of the formula I can either be synthesized building block by building block, or by initially constructing relatively large fragments consisting of several individual building blocks, which can then be joined to give the complete molecule.

Owing to the meanings which the individual building blocks of the compounds of the formula I can assume, ether [—O—], ester [—O—C(O)—], keto [—C(O)—], amide [—C(O)—NH—, —NH—C(O)—], carbamate [—NH—C(O)—O—, —O—C(O)—NH—], carbamide [—NH—C(O)—NH—] or carbonate [—O—C(O)—O—] bridges are present in the compounds of the formula I.

How to prepare such bridges is known per se to the person skilled in the art; suitable methods and starting materials for their preparation are described, for example, in March, Advanced Organic Chemistry, Reactions, Mechanisms and Structure, Third Edition, 1985, John Wiley & Sons.

Ether bridges can be prepared, for example, by the method of Williamson.

There is a large number of known methods for preparing ester bridges. An example which may be mentioned here is the reaction of acids with alcohols, preferably using H₂SO₄ or p-toluenesulfonic acid as catalyst; or with addition of a dehydrating agent, such as, for example, molecular sieve or a carbodiimide. Furthermore, the reaction of acyl chlorides with alcohols may be mentioned here.

Keto bridges can be introduced, for example, as a component of relatively large building blocks, such as, for example, carboxylic acid derivatives.

There is also a large number of known methods for preparing amide bridges. An example which may be mentioned here is the reaction of acyl chlorides with primary or secondary amines. Furthermore, reference is also made to all the methods which have been developed for peptide chemistry.

Carbamate bridges can be prepared, for example, by reacting chloroformates with amines. The chloroformates for their part can be synthesized from alcohols and phosgene. A further variant for constructing carbamate bridges is the addition of alcohols to isocyanates. Similarly to carbamate bridges, it is possible to prepare carbonate bridges starting from chloroformates, by reaction with alcohols (instead of amines).

Carbamide bridges can be prepared, for example, by reacting isocyanates with amines.

The preparation of compounds of the formula I may be shown in an exemplary manner using the reaction schemes below. The reaction scheme I shows the preparation of some starting compounds. The reaction schemes 2 to 4 show the preparation of exemplary compounds of formula I. Other compounds of the formula I can be prepared analogously, or by using the abovementioned methods known per se to the person skilled in the art.

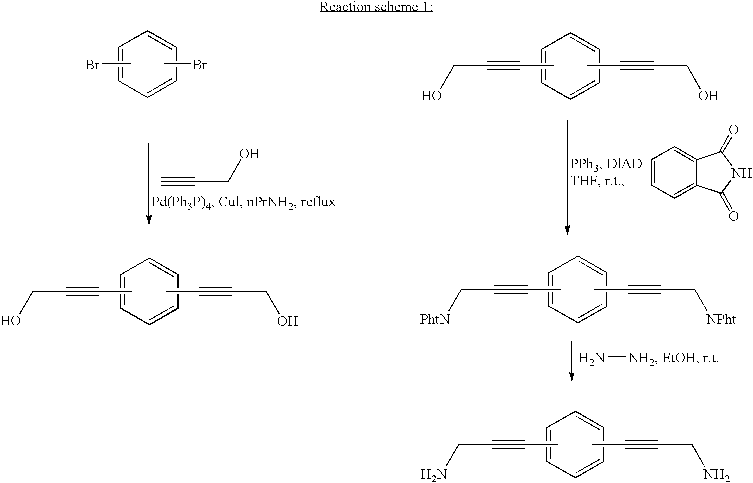

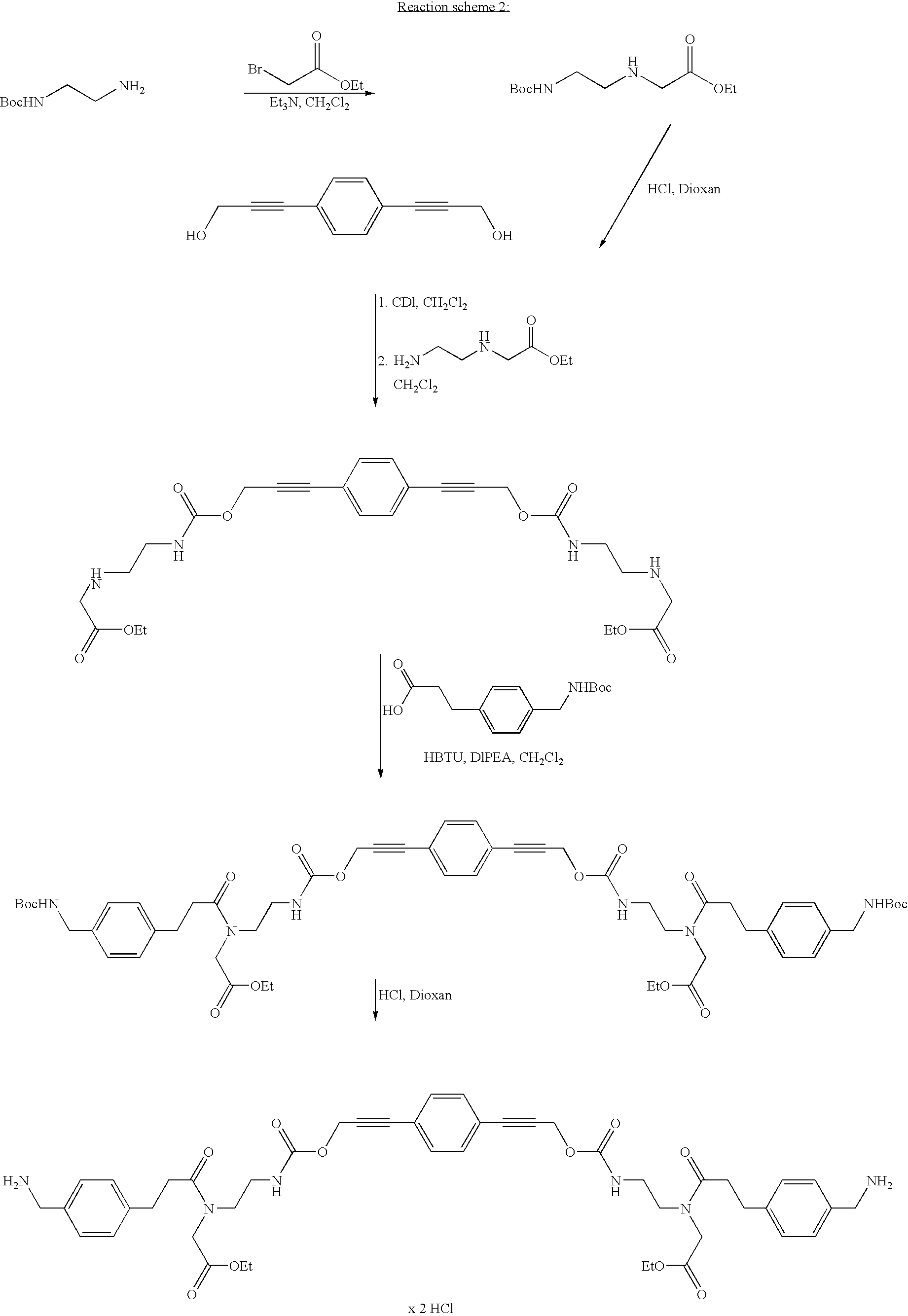

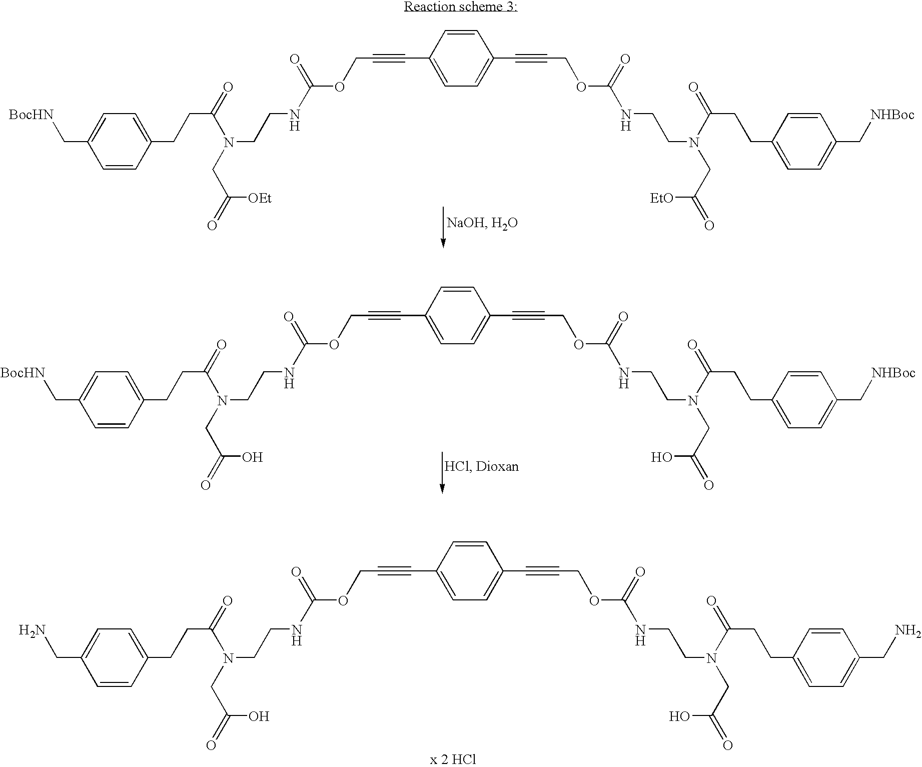

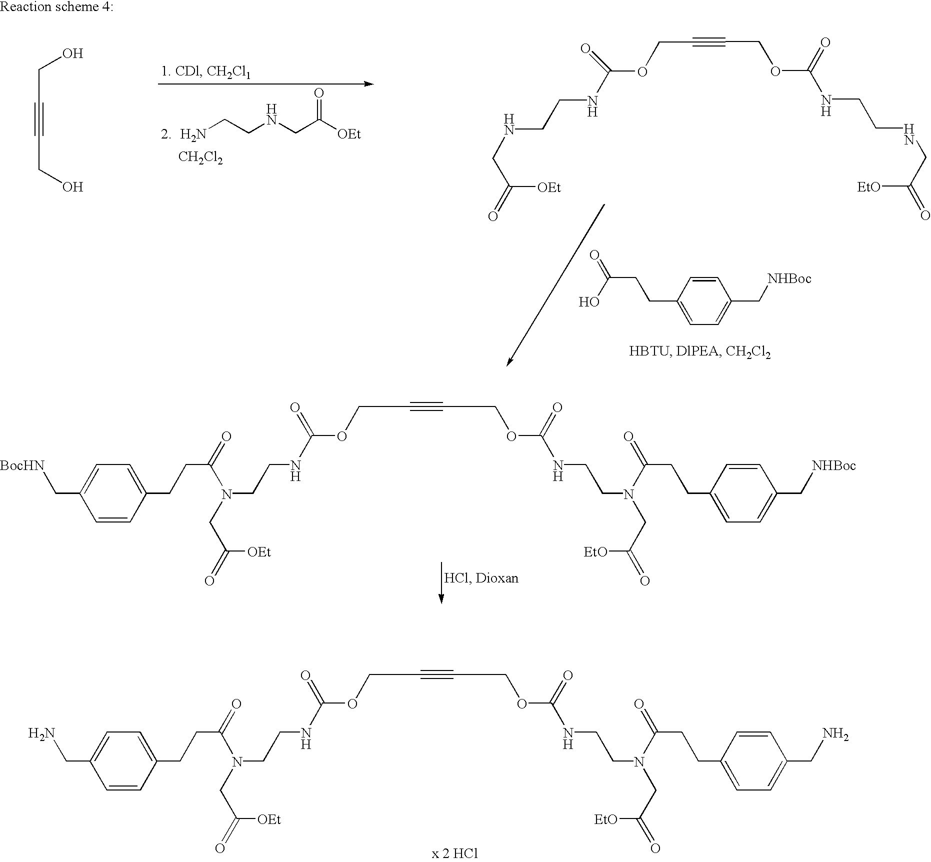

It is also possible to convert compounds of the formula I by derivatization into other compounds of the formula I. Thus, for example, compounds of the formula I having a nitrogen-containing heteroaryl, heteroarylene or heterocycloalkylene building block can be converted by oxidation into the corresponding N-oxides.

The N-oxidation is carried out in a manner which is likewise known to the person skilled in the art, for example using hydrogen peroxide in methanol or m-chloroperoxybenzoic acid in dichloromethane at room temperature. Which reaction conditions are required in the particular case for carrying out the process is known to the person skilled in the art owing to his expert knowledge.

It is furthermore known to the person skilled in the art that if there are a number of reactive centers on a starting material or intermediate, it may be necessary to block one or more reactive centers temporarily by protective groups in order to allow a reaction to proceed specifically at the desired reaction center. A detailed description of the use of a large number of proven protective groups is found, for example, in T. W. Greene, Protective Groups in Organic Synthesis, John Wiley & Sons, 1991.

The isolation and purification of the substances according to the invention is carried out in a manner known per se, for example by distilling off the solvent under reduced pressure and recrystallizing the resulting residue from a suitable solvent or subjecting it to one of the customary purification methods, such as, for example, column chromatography on a suitable support material.

Salts are obtained by dissolving the free compound in a suitable solvent (for example a ketone, such as acetone, methyl ethyl ketone or methyl isobutyl ketone, an ether, such as diethyl ether, tetrahydrofuran or dioxane, a chlorinated hydrocarbon, such as methylene chloride or chloroform, or a low-molecular-weight aliphatic alcohol, such as ethanol or isopropanol) which contains the desired acid or base, or to which the desired acid or base is then added. The salts are obtained by filtering, reprecipitating, precipitating with a nonsolvent for the addition salt or by evaporating the solvent. Salts obtained can be converted by alkalization or by acidification into the free compounds, which in turn can be converted into salts. In this way, pharmacologically unacceptable salts can be converted into pharmacologically acceptable salts.

The examples below serve to illustrate the invention in more detail without restricting it. Likewise, further compounds of the formula I, whose preparation is not explicitly described, can be prepared in an analogous manner or in a manner familiar per se to the person skilled in the art using customary process techniques.

In the examples below, the abbreviation RT stands for room temperature, h for hours, min. for minutes, m. p. for melting point, DIPEA for diisopropylethylamine, TLC stands for thin-layer chromatography and MS for mass spectrometry. The compounds mentioned by way of example and their salts are the preferred subject of the invention.

### EXAMPLES

### End Products

### General Procedure

A solution of the particular Boc-protected divalent compound (A4, A5, A7; 1.0 mmol) in dioxane (4 ml) is admixed with a saturated solution of HCl in dioxane (4 ml, 18.0 mmol) and stirred at RT for 4 h. The resulting precipitate is filtered off under an N₂ atmosphere and washed first with dioxane (2×5 ml) and then with diethyl ether (3×5 ml). Drying under reduced pressure gives the title compounds (end products 1-3) as colorless solids.

### 1. 1,4-Bis-{N-[3-(4-aminomethyl-phenyl)-propionyl]-N-(ethoxycarbonyl-methyl)-amino-3 -(ethyl-aminocarbonyloxy)-prop-1-ynyl}-benzene dihydrochloride

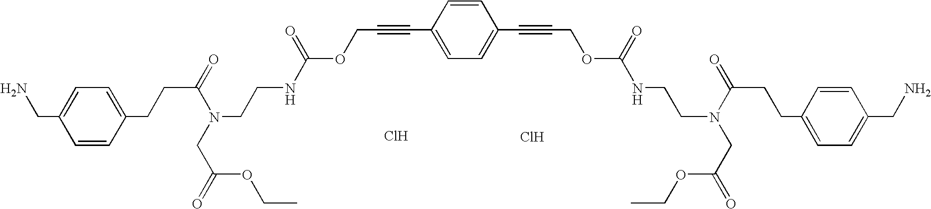

MS: calc.: C₄₈H₅₆N₆O₁₀ (852.99), found: [MH⁺] 853.3

### 2. 1,4-{Bis-N-[3-(4-(aminomethyl-phenyl)-propionyl]-N-(carboxymethyl)-amino-3-(ethyl-aminocarbonyloxy)-prop-1-ynyl}-benzene dihydrochloride

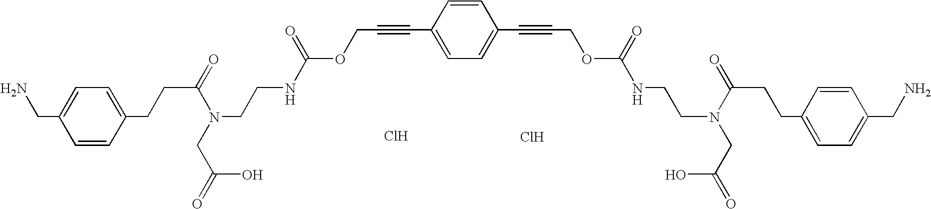

MS: calc.: C₄₂H₄₈N₆O₁₀ (796.7), found: [MH⁺] 797.2

### 3. 1,4-Bis{N-[3-(4-(aminomethyl-phenyl)-propionyl]-N-(ethoxycarbonyl-methyl)-amino-3-(ethyl-aminocarbonyloxy)}-2-butyne dihydrochloride

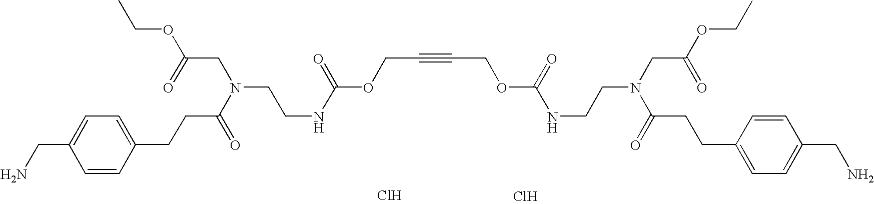

MS: calc.: C₃₈H₅₂N₆O₁₀ (752.9), found: [MH⁺] 753.3

### Starting Materials

### A1. (2-tert-Butoxycarbonylamino-ethylamino)-acetic acid ethylester

Ethylbromoacetat (320 μl, 2.0 mmol) is added to a solution of (2-Amino-ethyl)-carbamic acid tert-butylester (223 μl, 2.0 mmol) and Et₃N (310 μl, 2.2 mmol) in absolute CH₂Cl₂ (4 ml), and the mixture is stirred at 0° C. for 1 h. The reaction solution is diluted with CH₂Cl₂ (5 ml) and extracted with a semisaturated aqueous NaCl solution (10 ml). The organic phase is dried over MgSO₄, filtered off and concentrated under reduced pressure. Further purification by chromatography [Tol/Ac (8:2)] over a silica gel column gives the title compound (0.66 g) as a colorless oil. TLC, silica gel, glass plates, [Tol/Ac (8:2)] (7.5:2.5)], R_{f}=0.19.

MS: calc.: C₁₁H₂₂N₂O₄ (246.31), found: [MH⁺] 247.0; [MNa⁺] 269.0

### A2. (2-Amino-ethylamino)-acetic acid ethylester

A solution of (2-tert-Butoxycarbonylamino-ethylamino)-acetic acid ethylester (1.0 g; 4.0 mmol) in dioxane (5 ml) is admixed with a saturated solution of HCl in dioxane (4 ml, 18.0 mmol) and stirred at RT for 1 h. The resulting precipitate is filtered off under an N₂ atmosphere and washed first with dioxane (2×5 ml) and then with diethyl ether (3×5 ml). Drying under reduced pressure gives the title compound (0.9 g) as a colorless solid.

MS: calc.: C₆H₁₄N₂O₂ (146.2), found: [MH⁺] 147.0

### A3. 1,4-Bis-[N-(ethoxycarbonylmethyl)-amino-3-(ethyl-aminocarbonyloxy)-prop-1-ynyl]-benzene

N,N-carbonyldiimidazole (540 mg, 3.3 mmol) is added to a solution of 1,4-Bis-(3-Hydroxyprop-1-ynyl)-benzene (200 mg, 1.1 mmol) in absolute CH₂Cl₂ (7 ml), and the mixture is stirred at room temperature for 3 h. The reaction solution is diluted with CH₂Cl₂ (7 ml) and extracted with a semisaturated aqueous NaCl solution (15 ml). The organic phase is dried over MgSO₄, filtered off and concentrated under reduced pressure. The resulting residue is taken up in absolute CH₂Cl₂ (7 ml), (2-Amino-ethylamino)-acetic acid ethylester (322 mg, 2.2 mmol) is added and the mixture is stirred at room temperature overnight. The reaction solution is diluted with CH₂Cl₂ (7 ml) and extracted with a semisaturated aqueous NaCl solution (15 ml). The organic phase is dried over MgSO₄, filtered off and concentrated under reduced pressure. Further purification by chromatography [CH₂Cl₂/MeOH (95:0.5)] over a silica gel column gives the title compound (0.24 g) as a colorless solid. TLC, silica gel, glass plates [CH₂Cl₂/MeOH (95:0.5)], R_{f}=0.10.

MS: calc.: C₂₅H₃₄N₄O₈ (530.5), found: [MH⁺] 531.1

### A4. 1,4-Bis-{N-[3-(4-(tert-butoxycarbonylamino-methyl-phenyl)-propionyl]-N-(ethoxy-carbonyl-methyl)-amino-3-(ethyl-aminocarbonyloxy)-prop-1-ynyl}-benzene

O-(Benzotriazol-1-yl)-N,N,N′,N′-tetramethyluronium-hexafluorophosphate (HBTU, 0.37 g, 1.0 mmol) is added to a suspension of 3-[4-(tert-Butoxycarbonylamino-methyl)-phenyl]-propionic acid (0.28 g, 1.0 mmol) in absolute CH₂Cl₂ (4 ml) and DIPEA (0.23 ml), and the mixture is stirred at RT for 20 min. 1,4Bis-[N-(ethoxycarbonylmethyl)-amino-3-ethyl-aminocarbonyloxy)-prop-1ynyl]-benzene (0.24 g, 0.45 mmol) is then added, and the mixture is stirred at RT overnight. The reaction solution is diluted with CH₂Cl₂ (10 ml) and extracted (2×) with semisaturated aqueous NH₄Cl solution (15 ml), dried over MgSO₄, filtered off and concentrated under reduced pressure. Further purification by chromatography [Tol/Ac (7.5:2.5)] over a silica gel column gives the title compound (0.32 g) as a colorless solid. TLC, silica gel, glass plates, [Tol/Ac (7.5:2.5)], R₁=0.26.

MS: calc.: C₅₆H₇₂N₆O₁₄ (1052.0), found: [MH⁺] 1053.1; [MNa⁺] 1075.4

### A5. 1,4-Bis-{N-[3-(4-(tert-butoxycarbonylamino-methyl-phenyl)-propionyl]-N-(carboxy-methyl)-amino-3-(ethyl-aminocarbonyloxy)-prop-1-ynyl}-benzene

An aqueous solution of sodium hydroxide (1 ml, 5 N) is added to a solution of 1,4-Bis-{N-[3-(4-(tert-butoxycarbonylamino-methyl-phenyl)-propionyl]-N-(ethoxycarbonyl-methyl)-amino-3-(ethyl-aminocarbonyloxy)-prop-1-ynyl}-benzene (0.3 g, 0.18 mmol) in ethanol (3 ml). After stirring for 1 h at RT, an aqueous solution of KHSO₄ (20%) is added dropwise till pH=3. The reaction solution is diluted with CH₂Cl₂ (10 ml) and extracted (2×) with semisaturated aqueous NH₄Cl solution (15 ml), dried over MgSO₄, filtered off and concentrated under reduced pressure. Further purification by chromatography [CH₂Cl₂/MeOH (85:15)] over a silica gel column gives the title compound (0.32 g) as a colorless solid. TLC, silica gel, glass plates, [CH₂Cl₂/MeOH (85:15)], R_{f}=0.21.

MS: calc.: C₅₂H₆₄N₆O₁₄ (996.0), found: [MH⁺] 996.8; [MNH₄⁺] 1013.9, [MNa⁺] 1019.3

### A6. 1,4-Bis-[N-(ethoxycarbenylmethyl)-amino-3-(ethyl-aminocarbenyloxy)]-2-butyne

N,N-carbonyldiimidazole (1.72 g, 10.5 mmol) is added to a solution of But-2-yne-1,4-diol (300 mg, 3.5 mmol) in absolute CH₂Cl₂ (8 ml), and the mixture is stirred at room temperature for 2.5 h. The reaction solution is diluted with CH₂Cl₂ (8 ml) and extracted with a semisaturated aqueous NaCl solution (20 ml). The organic phase is dried over MgSO₄, filtered off and concentrated under reduced pressure. The resulting residue is taken up in absolute CH₂Cl₂ (8 ml), (2-Amino-ethylamino)-acetic acid ethylester (1.3 g, 8.9 mmol) is added and the mixture is stirred at room temperature overnight. The reaction solution is diluted with CH₂Cl₂ (8 ml) and extracted with a semisaturated aqueous NaCl solution (20 ml). The organic phase is dried over MgSO₄, filtered off and concentrated under reduced pressure. Further purification by chromatography [CH₂Cl₂/MeOH (95:0.5)] over a silica gel column gives the title compound (0.4 g) as a colorless solid. TLC, silica gel, glass plates [CH₂Cl₂/MeOH (95:0.5)], R_{f}=0.23.

MS: calc.: C₁₈H₃₀N₄O₈ (430.4), found: [MH⁺] 431.2

### A7. 1,4-Bis-{N-[3-(4-(tert-butoxycarbonyaminomethyl-phenyl)-propionyl]-N-(ethoxycarbonylmethyl)-amino-3-(ethyl-aminocarbonyloxy)}-2-butyne

O-(Benzotriazol-1-yl)-N,N,N′,N′-tetramethyluronium-hexafluorophosphate (HBTU, 0.76 g, 2.05 mmol) is added to a suspension of 3-[4-(tert-Butoxycarbonylamino-methyl)-phenyl]-propionic acid (0.56 g, 2.0 mmol) in absolute CH₂Cl₂ (10 ml) and DIPEA (0.48 ml), and the mixture is stirred at RT for 30 min. 1,4-Bis-[N-(ethoxycarbonylmethyl)-amino-3-(ethyl-aminocarbonyloxy)]-2-butyne (0.4 g, 0.93 mmol) is then added, and the mixture is stirred at RT overnight. The reaction solution is diluted with CH₂Cl₂ (10 ml) and extracted (2×) with semisaturated aqueous NH₄Cl solution (15 ml), dried over MgSO₄, filtered off and concentrated under reduced pressure. Further purification by chromatography [Tol/Ac (7:3)] over a silica gel column gives the title compound (0.3 g) as a colorless solid. TLC, silica gel, glass plates, [Tol/Ac (7:3)], R_{f}=0.28.

MS: calc.: C₄₈H₆₈N₆O₁₄ (952.0), found: [MH⁺] 953.0; [MNH₄⁺] 970.1, [MNa⁺975.4

### Commercial Utility

As tryptase inhibitors, the compounds according to the invention have useful pharmacological properties which make them commercially utilizable. Human tryptase is a serin protease which is the main protein in human mast cells. Tryptase comprises eight closely related enzymes (α1, α2, β1a, β1b, β2, β3, mMCP-7-like-1, mMCP-7-like-2; 85 to 99% sequence identity) (cf. Miller et al., J. Clin. Invest. 84 (1989) 1188-1195; Miller et al., J. Clin. Invest. 86 (1990) 864-870; Vanderslice et al., Proc. Natl. Acad. Sci., USA 87 (1990) 3811-3815; Pallaoro et al., J. Biol. Chem. 274 (1999) 3355-3362). However, only the β-tryptases (Schwartz et al., J. Clin. Invest. 96 (1995) 2702-2710; Sakai et al., J. Clin. Invest. 97 (1996) 988-995) are activated intracellularly and stored in catalytically active form in secretory granules. Compared with other known serin proteases, such as, for example, trypsin or chymotrypsin, tryptase has some special properties (Schwartz et al., Methods Enzymol. 244, (1994), 88-100; G. H. Caughey, “Mast cell proteases in immunology and biology”. Marcel Dekker, Inc., New York, 1995). Tryptase from human tissue has a noncovalently-linked tetrameric structure which has to be stabilized by heparin or other proteoglycanes to be proteolytically active. Together with other inflammatory mediators, such as, for example, histamine and proteoglycanes, tryptase is released when human mast cells are activated. Because of this, tryptase is thought to play a role in a number of disorders, in particular in allergic and inflammatory disorders, firstly because of the importance of the mast cells in such disorders and secondly since an increased tryptase concentration was observed in a number of disorders of this type. Thus, tryptase is associated, inter alia, with the following diseases: acute and chronic (in particular inflammatory and allergen-induced) airway disorders of various origins (for example bronchitis, allergic bronchitis, bronchial asthma, COPD); interstitial lung disorders; disorders based on allergic reactions of the upper airways, (pharynx, nose) and the adjacent regions (for example paranasal sinuses, conjunctivae), such as, for example allergic conjunctivitis and allergic rhinitis; disorders of the arthritis type (for example rheumatoid arthritis); autoimmune disorders, such as multiple sclerosis; furthermore periodontitis, anaphylaxis, interstitial cystitis, dermatitis, psoriasis, sclerodermia/systemic sclerosis, inflammatory intestinal disorders (Crohn's disease, Ulcerative Colitis) and others. In particular, tryptase seems to be connected directly to the pathogenesis of asthma (Caughey, Am. J. Respir. Cell Mol. Biol. 16 (1997), 621-628; R. Tanaka, “The role of tryptase in allergic inflammation” in: Protease Inhibitors, IBC Library Series, 1979, Chapter 3.3.1-3.3.23).

A further subject of the invention relates to the compounds according to the invention for use in the treatment and/or prophylaxis of diseases, in particular the diseases mentioned.

The invention likewise relates to the use of the compounds according to the invention for preparing medicaments which are employed for the treatment and/or prophylaxis of the diseases mentioned.

Medicaments for the treatment and/or prophylaxis of the diseases mentioned, which contain one or more of the compounds according to the invention, are furthermore a subject of the invention.

The medicaments are prepared by processes which are known per se and familiar to the person skilled in the art. As medicaments, the compounds according to the invention (=active compounds) are either employed as such, or preferably in combination with suitable pharmaceutical excipients, for example in the form of tablets, coated tablets, capsules, suppositories, patches, emulsions, suspension, gels or solutions, the active compound content advantageously being between 0.1 and 95%.

The person skilled in the art is familiar on the basis of his/her expert knowledge with the excipients which are suitable for the desired pharmaceutical formulations. In addition to solvents, gel-forming agents, ointment bases and other active compound vehicles, it is possible to use, for example, antioxidants, dispersants, emulsifiers, preservatives, solubilizers or permeation promoters.

For the treatment of disorders of the respiratory tract, the compounds according to the invention can be also administered by inhalation in the form of an aerosol; the aerosol particles of solid, liquid or mixed composition preferably having a diameter of 0.5 to 10 μm, advantagously of 2 to 6 μm.

Aerosol generation can be carried out, for example, by pressure-driven jet atomizers or ultrasonic atomizers, but advantageously by propellant-driven metered aerosols or propellant-free administration of micronized active compounds from inhalation capsules.

Depending on the inhaler system used, in addition to the active compounds the administration forms additionally contain the required excipients, such as, for example, propellants (e.g. Frigen in the case of metered aerosols), surface-active substances, emulsifiers, stabilizers, preservatives, flavorings, fillers (e.g. lactose in the case of powder inhalers) or, if appropriate, further active compounds.

For the purposes of inhalation, a large number of apparatuses are available with which aerosols of optimum particle size can be generated and administered, using an inhalation technique which is as right as possible for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhaler described in European Patent Application EP 0 505 321), using which an optimal administration of active compound can be achieved.

For the treatment of dermatoses, the compounds according to the invention are in particular used in the form of those medicaments which are suitable for topical administration. For the preparation of the medicaments, the compounds according to the invention (=active compounds) are preferably mixed with suitable pharmaceutical excipients and further processed to give suitable pharmaceutical formulations. Suitable pharmaceutical formulations which may be mentioned are, for example, powders, emulsions, suspensions, sprays, oils, ointments, fatty ointments, creams, pastes, gels or solutions.

The medicaments according to the invention are prepared by processes known per se. The dosage of the active compounds in the case of systemic therapy (p.o. or i.v.) is between 0.1 and 10 mg per kilogram per day.

### Biological Investigations

The documented pathophysiological effects of mast cell tryptase are caused directly by the enzymatic activity of the protease. Accordingly, they are reduced or blocked by inhibitors which inhibit the enzymatic activity of the tryptase. A suitable measure for the affinity of a reversible inhibitor to the target protease is the equilibrium dissociation constant Kᵢ of the enzyme-inhibitor complex. This Kᵢ value can be determined via the effect of the inhibitor on the tryptase-induced cleavage of a chromogenic peptide-p-nitroanilide substrate or a fluorogenic peptide-aminomethylcoumarin substrate.

### Methodology

The dissociation constants for the tryptase-inhibitor complexes are determined under equilibrium conditions in accordance with the general proposals of Bieth (Bieth J G, Pathophysiological Interpretation of kinetic constants of protease inhibitors, Bull. Europ. Physiopath. Resp. 16:183-195, 1980) and the methods of Sommerhoff et al. (Sommerhoff C P et al., A Kazal-type inhibitor of human mast cell tryptase: Isolation from the medical leech Hirudo medicinalis, characterization, and sequence analysis, Biol. Chem. Hoppe-Seyler 375: 685-694, 1994).

Human tryptase is isolated from lung tissue or prepared recombinantly; the specific activity of the protease, determined by titration, is usually greater than 85% of the theoretical value. In the presence of heparin (0.1-50 μg/ml) for stabilizing the protease, constant amounts of the tryptase are incubated with increasing amounts of the inhibitors. After an equilibrium between the reaction partners has formed, the remaining enzyme activity after addition of the peptide-p-nitroanilide substrate tos-Gly-Pro-arg-pNA is determined and the cleavage of the latter is monitored at 405 nm for 3 min. Alternatively, the remaining enzymatic activity can also be determined using fluorogenic substrates. The apparent dissociation constants Kᵢₐₚₚ, (i.e. in the presence of substrate) are subsequently determined by adapting the enzyme rates to the general equation for reversible inhibitors (Morrison J F, Kinetics of the reversible inhibition of enzyme-catalyzed reactions by tight-binding inhibitors, Biochim. Biophys. Acta 185, 269-286, 1969) using non-linear regression:

Vₗ/V₀=1−{Eₜ+Iₜ+Kᵢₐₚₚ−[(Eₜ+Iₜ+Kᵢₐₚₚ)²−4EₜIₜ]^{1/2}}/2Eₜ

Vₗ and V₀ are the rates in the presence and absence, respectively, of the inhibitor, and Eₜ and Iₜ are the tryptase and inhibitor concentrations, respectively.

The apparent dissociation constants determined for the compounds according to the invention are shown in Table A below, where the numbers of the compounds correspond to the numbers of the compounds in the examples.

TABLE A
Inhibition of human tryptase

| Compound | Kₗₐₚₚ (μM) |
|---|---|
| 1 | 0.035 |
| 3 | 0.0054 |

1. Compounds of formula I 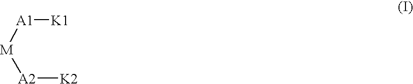
in which
M is a central building block selected from the formulae below 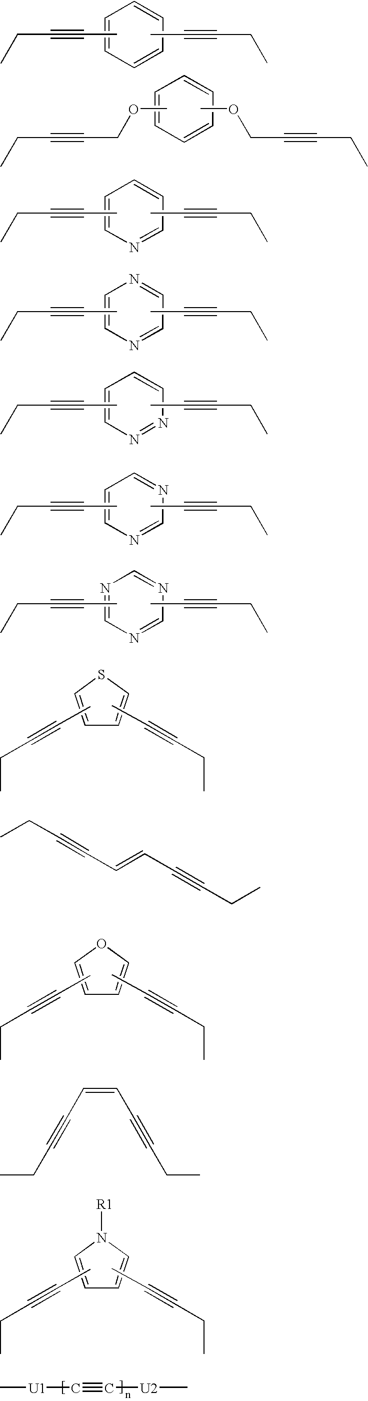
wherein
R1 is hydrogen, 1-4C-alkyl or 1-4C-alkylcarbonyl,
n is 1 or 2,
U1 and U2 are identical or different and are methylene [—CH₂—], ethylene [—CH₂—CH₂—], trimethylene [—CH₂—CH₂—CH₂—], tetramethylene [—CH₂—CH₂—CH₂—CH₂—] or isopropylidene [—C(CH₃)₂—],
A1 is -A3-B1-A5-,
A2 is -A4-B2-A6-,
wherein either
A3 is —C(O)—, —O—C(O)—, —C(O)—NH—, —NH—C(O)—, —O—C(O)—NH—, —NH—C(O)—O—, —NH—C(O)—NH—, —O—C(O)—O—, —O—(CH₂)ᵣ—C(O)—, —O—(CH₂)ᵣ—C(O)—NH—, —O—(CH₂)ₘ—O—C(O)— or —O—(CH₂)ₘ—NH—C(O)—,
A4 is —C(O)—, —O—C(O)—, —C(O)—NH—, —NH—C(O)—, —O—C(O)—NH—, —NH—C(O)—O—, —NH—C(O)—NH—, —O—C(O)—O—, —O—(CH₂)ᵣ—C(O)—, —O—(CH₂)ᵣ—C(O)—NH—, —O—(CH₂)ₘ—O—C(O)— or —O—(CH₂)ₘ—NH—C(O)—,
A5 is —C(O)—N(R2)-, —N(R2)-C(O)—, —O—C(O)—N(R2)- or —N(R2)-C(O)—O—, and
A6 is —C(O)—N(R3)-, —N(R3)-C(O)—, —O—C(O)—N(R3)- or —N(R3)-C(O)—O—,
or
A3 is —C(O)—N(R4)-, —N(R4)-C(O)—, —O—C(O)—N(R4)-, —N(R4)-C(O)—O—, —O—(CH₂)ᵣ—C(O)—N(R4)- or —O—(CH₂)ₘ—N(R4)-C(O)—,
A4 is —C(O)—N(R5)-, —N(R5)-C(O)—, —O—C(O)—N(R5)-, —N(R5)-C(O)—O—, —O—(CH₂)ᵣ—C(O)—N(R5)- or —O—(CH₂)ₘ—N(R5)-C(O)—,
A5 is —C(O)—, —C(O)—NH—, —NH—C(O)—, —O—C(O)—NH—, —NH—C(O)—O—, —NH—C(O)—NH— or —O—C(O)—O—, and
A6 is —C(O)—, —C(O)—NH—, —NH—C(O)—, —O—C(O)—NH—, —NH—C(O)—O—, —NH—C(O)—NH— or —O—C(O)—O—,
or
A3 is —C(O)—N(R4)-, —N(R4)-C(O)—, —O—C(O)—N(R4)-, —N(R4)-C(O)—O—, —O—(CH₂)ᵣ—C(O)—N(R4)- or —O—(CH₂)ₘ—N(R4)-C(O)—,
A4 is —C(O)—, —O—C(O)—, —C(O)—NH—, —NH—C(O)—, —O—C(O)—NH—, —NH—C(O)—O—, —NH—C(O)—NH—, —O—C(O)—O—, —O—(CH₂)ᵣ—C(O)—, —O—(CH₂)ᵣ—C(O)—NH—, —O—(CH₂)ₘ—O—C(O)— or —O—(CH₂)ₘ—NH—C(O)—,
A5 is —C(O)—N(R2)-, —N(R2)-C(O)—, —O—C(O)—N(R2)- or —N(R2)-C(O)—O—, and
A6 is —C(O)—, —C(O)—NH—, —NH—C(O)—, —O—C(O)—NH—, —NH—C(O)—O—, —NH—C(O)—NH— or —O—C(O)—O—,
or
A3 is —C(O)—, —O—C(O)—, —C(O)—NH—, —NH—C(O)—, —O—C(O)—NH—, —NH—C(O)—O—, —NH—C(O)—NH—, —O—C(O)—O—, —O—(CH₂)ᵣ—C(O)—, —O—(CH₂)ᵣ—C(O)—NH—, —O—(CH₂)ₘ—O—C(O)— or —O—(CH₂)ₘ—NH—C(O)—,
A4 is —C(O)—N(R5)-, —N(R5)-C(O)—, —O—C(O)—N(R5)-, —N(R5)-C(O)—O—, —O—(CH₂)ᵣ—C(O)—N(R5)- or —O—(CH₂)ₘ—N(R5)-C(O)—,
A5 is —C(O)—, —C(O)—NH—, —NH—C(O)—, —O—C(O)—NH—, —NH—C(O)—O—, —NH—C(O)—NH— or —O—C(O)—O—, and
A6 is —C(O)—N(R3)-, —N(R3)-C(O)—, —O—C(O)—N(R3)- or —N(R3)-C(O)—O—,
or
A3 is —C(O)—N(R4)-, —N(R4)-C(O)—, —O—C(O)—N(R4)-, —N(R4)-C(O)—O—, —O—(CH₂)ᵣ—C(O)—N(R4)- or —O—(CH₂)ₘ—N(R4)-C(O)—,
A4 is —C(O)—, —O—C(O)—, —C(O)—NH—, —NH—C(O)—, —O—C(O)—NH—, —NH—C(O)—O—, —NH—C(O)—NH—, —O—C(O)—O—, —O—(CH₂)ᵣ—C(O)—, —O—(CH₂)ᵣ—C(O)—NH—, —O—(CH₂)ₘ—O—C(O)— or —O—(CH₂)ₘ—NH—C(O)—,
A5 is —C(O)—, —C(O)—NH—, —NH—C(O)—, —O—C(O)—NH—, —NH—C(O)—O—, —NH—C(O)—NH— or —O—C(O)—O—, and
A6 is —C(O)—N(R3)-, —N(R3)-C(O)—, —O—C(O)—N(R3)- or —N(R3)-C(O)—O—,
or
A3 is —C(O)—, —O—C(O)—, —C(O)—NH—, —NH—C(O)—, —O—C(O)—NH—, —NH—C(O)—O—, —NH—C(O)—NH—, —O—C(O)—O—, —O—(CH₂)ᵣ—C(O)—, —O—(CH₂)ᵣ—C(O)—NH—, —O—(CH₂)ₘ—O—C(O)— or —O—(CH₂)ₘ—NH—C(O)—,
A4 is —C(O)—N(R5)-, —N(R5)-C(O)—, —O—C(O)—N(R5)-, —N(R5)-C(O)—O—, —O—(CH₂)ₘ—C(O)—N(R5)- or —O—(CH₂)ₘ—N(R5)-C(O)—,
A5 is —C(O)—N(R2)-, —N(R2)-C(O)—, —O—C(O)—N(R2)- or —N(R2)-C(O)—O—, and
A6 is —C(O)—, —C(O)—NH—, —NH—C(O)—, —O—C(O)—NH—, —NH—C(O)—O—, —NH—C(O)—NH— or —O—C(O)—O—,
and
r is 1, 2, 3 or 4,
m is 1, 2, 3or4,
R2, R3, R4 and R5 are identical or different and are —CH₂—C(O)OR6 or —CH₂—C(O)N(R7)R8,
R6 is hydrogen, 1-4C-alkyl, 3-7C-cycloalkyl, 3-7C-cycloalkylmethyl or benzyl,
R7 and R8 are independent from each other hydrogen, 1-4C-alkyl, 3-7C-cycloalkyl, 3-7C-cycloalkylmethyl, or wherein R7 and R8 together and with inclusion of the nitrogen atom to which they are bonded form a 1-pyrrolidinyl-, 1-piperidinyl-, 1-hexahydroazepinyl-, 1-piperazinyl- or 4-morpholinyl-radical,
B1 and B2 are identical or different and are 1-4C-alkylene, 1,4-cyclohexylene, 1,3-cyclohexylene, 1,4-phenylene, 1,3-phenylene, 1,4-piperazinylene or 1,4-piperidinylene,
K1 is -B3-X1, -B3-Y1 or -B3-Z1-B5-X1,
K2 is -B4-X2, -B4-Y2 or -B4-Z2-B6-X2,
B3 and B4 are identical or different and are a bond or 1-4C-alkylene,
B5 and B6 are identical or different and are a bond or 1-2C-alkylene,
X1 and X2 are identical or different and are amino, aminocarbonyl or amidino,
Y1 and Y2 are imidazol-1-yl,
Z1 and Z2 are identical or different and are 5,2-pyridinylene, 6-methyl-5,2-pyridinylene, 4,1-piperidinylene, 3,6-indazolylene, 3,6-indolylene, 1,3-phenylene, 1,4-phenylene, 1,3-cyclohexylene or 1,4-cyclohexylene,
the salts of these compounds, and the N-oxides of the nitrogen-containing heteroaryls, heteroarylenes and heterocycloalkylenes, and their salts, where all those compounds are excluded in which, owing to the meaning of the variables A3, A4, A5, A6, B1, B2, B3, B4, Y1, Y2, Z1, Z2, X1 or X2, there would be a direct linkage of two heteroatoms or two carbonyl groups.

2. Compounds of formula I according to claim 1, in which
M is a central building block selected from the formulae below 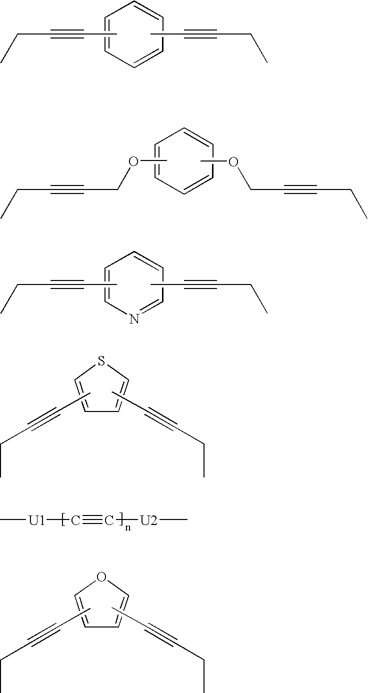
wherein
n is 1 or 2,
U1 and U2 are identical or different and are methylene [—CH₂—], ethylene [—CH₂—CH₂—], trimethylene [—CH₂—CH₂—CH₂—], tetramethylene [—CH₂—CH₂—CH₂—CH₂—] or isopropylidene [—C(CH₃)₂—],
A1 is -A3-B1-A5-,
A2 is -A4-B2-A6-,
wherein either
A3 is —O—C(O)—, —NH—C(O)—, —O—C(O)—NH—, —NH—C(O)—O—, —NH—C(O)—NH—, —O—C(O)—O—, —O—(CH₂)ᵣ—C(O)—, —O—(CH₂)ᵣ—C(O)—NH—, —O—(CH₂)ₘ—O—C(O)— or —O—(CH₂)ₘ—NH—C(O)—,
A4 is —O—C(O)—, —NH—C(O)—, —O—C(O)—NH—, —NH—C(O)—O—, —NH—C(O)—NH—, —O—C(O)—O—, —O—(CH₂)ᵣ—C(O)—, —O—(CH₂)ᵣ—C(O)—NH—, —O—(CH₂)ₘ—O—C(O)— or —O—(CH₂)ₘ—NH—C(O)—,
A5 is —C(O)—N(R2)- or —N(R2)-C(O)—, and
A6 is —C(O)—N(R3)- or —N(R3)-C(O)—,
or
A3 is —N(R4)-C(O)—, —O—C(O)—N(R4)-, —N(R4)-C(O)—O—, —O—(CH₂)ᵣ—C(O)—N(R4)- or —O—(CH₂)ₘ—N(R4)-C(O)—,
A4 is —N(R5)-C(O)—, —O—C(O)—N(R5)-, —N(R5)-C(O)—O—, —O—(CH₂)ᵣ—C(O)—N(R5)- or —O—(CH₂)ₘ—N(R5)-C(O)—,
A5 is —C(O)—, —C(O)—NH— or —NH—C(O)—, and
A6 is —C(O)—, —C(O)—NH— or —NH—C(O)—,
or
A3 is —C(O)—N(R4)-, —N(R4)-C(O)—, —O—C(O)—N(R4)-, —N(R4)-C(O)—O—, —O—(CH₂)ᵣ—C(O)—N(R4)- or —O—(CH₂)ₘ—N(R4)-C(O)—,
A4 is —O—C(O)—, —NH—C(O)—, —O—C(O)—NH—, —NH—C(O)—O—, —NH—C(O)—NH—, —O—C(O)—O—, —O—(CH₂)ᵣ—C(O)—, —O—(CH₂)ᵣ—C(O)—NH—, —O—(CH₂)ₘ—O—C(O)— or —O—(CH₂)ₘ—NH—C(O)—,
A5 is —C(O)—N(R2)- or —N(R2)-C(O)—, and
A6 is —C(O)—, —C(O)—NH— or —NH—C(O)—,
or
A3 is —O—C(O)—, —NH—C(O)—, —O—C(O)—NH—, —NH—C(O)—O—, —NH—C(O)—NH—, —O—C(O)—O—, —O—(CH₂)ᵣ—C(O)—, —O—(CH₂)ᵣ—C(O)—NH—, —O—(CH₂)ₘ—O—C(O)— or —O—(CH₂)ₘ—NH—C(O)—,
A4 is —N(R5)-C(O)—, —O—C(O)—N(R5)-, —N(R5)-C(O)—O—, —O—(CH₂)ᵣ—C(O)—N(R5)- or —O—(CH₂)ₘ—N(R5)-C(O)—,
A5 is —C(O)—, —C(O)—NH— or —NH—C(O)—, and
A6 is —C(O)—N(R3)- or —N(R3)-C(O)—,
or
A3 is —N(R4)-C(O)—, —O—C(O)—N(R4)-, —N(R4)-C(O)—O—, —O—(CH₂)ᵣ—C(O)—N(R4)- or —O—(CH₂)ᵣ—N(R4)-C(O)—,
A4 is —O—C(O)—, —NH—C(O)—, —O—C(O)—NH—, —NH—C(O)—O—, —NH—C(O)—NH—, —O—C(O)—O—, —O—(CH₂)ᵣ—C(O)—, —O—(CH₂)ᵣ—C(O)—NH—, —O—(CH₂)ₘ—O—C(O)— or —O—(CH₂)ₘ—NH—C(O)—,
A5 is —C(O)—, —C(O)—NH— or —NH—C(O)—, and
A6 is —C(O)—N(R3)- or —N(R3)-C(O)—,
or
A3 is —O—C(O)—, —NH—C(O)—, —O—C(O)—NH—, —NH—C(O)—O—, —NH—C(O)—NH—, —O—C(O)—O—, —O—(CH₂)ᵣ—C(O)—, —O—(CH₂)ᵣ—C(O)—NH—, —O—(CH₂)ₘ—O—C(O)— or —O—(CH₂)ₘ—NH—C(O)—,
A4 is —N(R5)-C(O)—, —O—C(O)—N(R5)-, —N(R5)—C(O)—O—, —O—(CH₂)ᵣ—C(O)—N (R5)- or —O—(CH₂)ₘ—N(R5)-C(O)—,
A5 is —C(O)—N(R2)- or —N(R2)-C(O)—, and
A6 is —C(O)—, —C(O)—NH— or —NH—C(O)—,
and
r is 1 or 2,
m is 2,
R2, R3, R4 and R5 are identical or different and are —CH₂—C(O)OR6 or —CH₂—C(O)N(R7)R8,
R6 is hydrogen, 1-4C-alkyl, 3-7C-cycloalkyl, 3-7C-cycloalkylmethyl or benzyl,
R7 and R8 are independent from each other hydrogen, 1-4C-alkyl, 3-7C-cycloalkyl, 3-7C-cycloalkylmethyl, or wherein R7 and R8 together and with inclusion of the nitrogen atom to which they are bonded form a 1-pyrrolidinyl-, 1-piperidinyl-, 1-hexahydroazepinyl-, 1-piperazinyl- or 4-morpholinyl-radical,
B1 and B2 are identical or different and are 1-4C-alkylene, 1,4-cyclohexylene, 1,3-cyclohexylene, 1,4-phenylene, 1,3-phenylene, 1,4-piperazinylene or 1,4-piperidinylene,
K1 is -B3-Z1-B5-X1,
K2 is -B4-Z2-B6-X2,
B3 and B4 are Identical or different and are a bond or 1-2C-alkylene,
B5 and B6 are identical or different and are a bond or 1-2C-alkylene,
X1 and X2 are identical or different and are amino or amidino,
Z1 and Z2 are identical or different and are 1,3-phenylene, 1,4-phenylene, 1,3-cyclohexylene or 1,4-cyclohexylene,
and the salts of these compounds, and the N-oxides of the nitrogen-containing heterocycloalkylenes, and their salts, where all those compounds are excluded in which, owing to the meaning of the variables A3, A4, A5, A6, B1 or B2, there would be a direct linkage of two heteroatoms.

3. Compounds of formula I according to claim 1 in which
M is a central building block selected from the formulae below 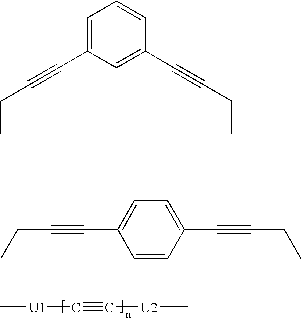
wherein
n is 1 or 2,
U1 and U2 are identical and are methylene [—CH₂—],
A1 is -A3-B1-A5-,
A2 is -A4-B2-A6-,
wherein either
A3 is —O—C(O)—NH—,
A4 is —O—C(O)—NH—,
A5 is —C(O)—N(R2)- or —N(R2)-C(O)—, and
A6—C(O)—N(R3)- or —N(R3)-C(O)—,
or
A3 is —O—C(O)—N(R4)-,
A4 is —O—C(O)—N(R5)-,
A5 is —C(O)—NH— or —NH—C(O)—, and
A6 is —C(O)—NH— or —NH—C(O)—,
or
A3 is —O—C(O)—N(R4)-,
A4 is —O—C(O)—NH—,
A5 is —C(O)—N(R2)- or —N(R2)-C(O)—, and
A6 is —C(O)—NH— or —NH—C(O)—,
or
A3 is —O—C(O)—NH—,
A4 is —O—C(O)—N(R5)-,
A5 is —C(O)—NH— or —NH—C(O)—, and
A6 is —C(O)—N(R3)- or —N(R3)-C(O)—,
or
A3 is —O—C(O)—N(R4)-,
A4 is —O—C(O)—NH—,
A5 is —C(O)—NH— or —NH—C(O)—, and
A6 is —C(O)—N(R3)- or —N(R3)-C(O)—,
or
A3 is —O—C(O)—NH—,
A4 is —O—C(O)—N(R5)-,
A5 is —C(O)—N(R2)- or —N(R2)-C(O)—, and
A6 is —C(O)—NH— or —NH—C(O)—,
and
R2, R3, R4 and R5 are identical and are —CH₂—C(O)OR6,
R6 is hydrogen, 1-4C—alkyl or benzyl,
B1 and B2 are identical and are ethylene,
K1 is -B3-Z1-B5-X1,
K2 is -B4-Z2-B6-X2,
B3 and B4 are identical and are ethylene,
B5 and B6 are identical and are methylene,
X1 and X2 are identical and are amino,
Z1 and Z2 are identical and are 1,3-phenylene or 1,4-phenylene,
and the salts of these compounds.

4. Compounds of formula I according to claim 1 in which
M is a central building block selected from the formulae below 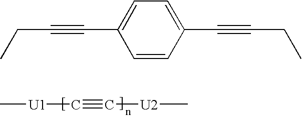
wherein
n is 1,
U1 and U2 are identical and are methylene [—CH₂—],
A1 is -A3-B1-A5-,
A2 is -A4-B2-A6-,
wherein
A3 is —O—C(O)—NH—,
A4 is —O—C(O)—NH—,
A5 is —N(R2)-C(O)—,
A6 is —N(R3)-C(O)—,
R2 and R3 are identical and are —CH₂—C(O)OR6, and
R6 is 1-2C-alkyl,
B1 and B2 are identical and are ethylene,
K1 is -B3-Z1-B5-X1,
K2 is -B4-Z2-B6-X2,
B3 and B4 are identical and are ethylene,
B5 and B6 are identical and are methylene,
X1 and X2 are identical and are amino,
Z1 and Z2 are identical and are 1,4-phenylene,
and the salts of these compounds.

5. Compounds of formula I according to claim 1 with the chemical name 1,4-Bis-{N-[3-(4-aminomethyl-phenyl)-propionyl]-N-(ethoxycarbonyl-methyl)-amino-3-(ethyl-aminocarbonyloxy)-prop-1-ynyl}-benzene, 1,4-Bis-{N-[3-(4-(aminomethyl-phenyl)-propionyl]-N-(carboxymethyl)-amino-3-(ethyl-aminocarbonyloxy)-prop-1-ynyl}-benzene, 1,4-Bis-{N-[3-(4-(aminomethyl-phenyl)-propionyl]-N-(ethoxy-carbonyl-methyl)-amino-3-(ethyl-aminocarbonyloxy)}-2butyne, and the salts of these compounds.

6. Compounds of formula I according to claim 1 with the chemical name 1,4-Bis-{N-[3-(4-aminomethyl-phenyl)-propionyl]-N-(ethoxycarbonyl-methyl)-amino-3-(ethyl-aminocarbonyloxy)-prop-1-ynyl}-benzene, 1,4-Bis-{N-[3-(4-(aminomethyl-phenyl)-propionyl]-N-(ethoxycarbonyl-methyl)-amino-3-(ethyl-aminocarbonyloxy)}-2-butyne, and the salts of these compounds.

7. Compounds of formula I according to claim 1 for use in the treatment of diseases.

8. A medicament comprising one or more compounds of formula I according to claim 1 together with customary pharmaceutical auxiliaries and/or excipients.

9. Use of compounds of formula I according to claim 1 for the production of medicaments for the treatment of airway disorders.
